(19) United States
(12) Patent Application Publication
(10) Pub. No.: US 2024/0032950 A1
(43) Pub. Date: Feb. 1, 2024

(54) FLUID PERFUSION APPARATUS AND FLUID PERFUSION METHOD

(71) Applicant: OLYMPUS MEDICAL SYSTEMS CORP., Tokyo (JP)

(72) Inventors: Nagahide SAKAI, Tokyo (JP); Satoshi HORIE, Tokyo (JP)

(73) Assignee: OLYMPUS MEDICAL SYSTEMS CORP., Tokyo (JP)

(21) Appl. No.: 18/211,688

(22) Filed: Jun. 20, 2023

Related U.S. Application Data
(60) Provisional application No. 63/356,055, filed on Jun. 28, 2022.

(51) Int. Cl. A61B 17/22

(52) U.S. Cl. CPC A61B 17/22; A61B 2017/22079; A61B 2217/005; A61B 2017/00022

(57) ABSTRACT

A fluid perfusion apparatus includes: a suction conduit for suctioning a fluid from inside a living body; a suction source connected to the suction conduit and configured to suction the fluid at a first flow velocity via the suction conduit; a suction control apparatus connected to the suction conduit and configured to control a flow of the fluid suctioned via the suction conduit; and a control circuit configured to control the suction control apparatus. The control circuit performs control for reversing the flow of the fluid suctioned via inside the suction conduit by the suction control apparatus, to generate a backflow, after controlling suction at the first flow velocity by the suction source for a predetermined period of time and to cause the suction control apparatus to perform re-suction at a second flow velocity greater than the first flow velocity after continuing the backflow for the predetermined period of time.

### RELATED APPLICATION DATA

This application is based on and claims priority under 35 U.S.C. § 119 to U.S. Provisional Application No. 63/356,055 filed on Jun. 28, 2022, the entire contents of which are incorporated herein by reference.

### FIELD

The present invention relates to a fluid perfusion apparatus and a fluid perfusion method.

### BACKGROUND

Conventionally, as an apparatus for collecting stones from inside a body, a stone collection apparatus which fragments a stone using laser light and collects fragmented stone pieces has been developed. For example, a technique for fragmenting stones by emitting laser light from a laser probe inserted into a treatment instrument channel of an endoscope has been proposed. In the proposal, the fragmented stones (crushed stones) are grasped with forceps and excised to the outside of the body.

In addition, there is also a stone treatment system which collects stones by performing water feeding and suction. In the system, a stone is perfused together with water via a suction pipe and collected outside of the body. However, the stone may become trapped in the suction pipe. In such a case, the trapped stone may act as a starting point to trap subsequent stones and may eventually lead to obstruction of the suction pipe.

In consideration thereof, International Publication No. 2023/026447 discloses a technique for preventing a suction pipe from reaching obstruction by detecting a perfusion state of a conduit and controlling a flow of a liquid in the conduit based on a result of the detection of the perfusion state.

### SUMMARY OF THE INVENTION

A fluid perfusion apparatus according to an aspect of the present invention includes: a suction conduit for suctioning a fluid from inside a living body; a suction source connected to the suction conduit and configured to suction the fluid at a first flow velocity via the suction conduit; a suction control apparatus connected to the suction conduit and configured to control a flow of the fluid suctioned via the suction conduit; and a control circuit configured to control the suction control apparatus, wherein the control circuit is configured to perform control for reversing the flow of the fluid that is suctioned via inside the suction conduit by the suction control apparatus, to generate a backflow, after controlling suction at the first flow velocity by the suction source for a predetermined period of time and to cause the suction control apparatus to perform re-suction at a second flow velocity that is greater than the first flow velocity after continuing the backflow for the predetermined period of time.

In addition, a fluid perfusion apparatus according to another aspect of the present invention includes: a suction conduit for suctioning a fluid from inside a living body; a suction source connected to the suction conduit and configured to suction the fluid at a first flow velocity via the suction conduit; a suction control apparatus connected to the suction conduit and configured to control a flow of the fluid suctioned via the suction conduit; a fluid detection apparatus configured to detect a flow rate of the fluid that flows through the suction conduit or an internal pressure of the suction conduit; and a control circuit configured to control the suction control apparatus based on information from the fluid detection apparatus, wherein the control circuit is configured to perform control for reversing the flow of the fluid that is suctioned via inside the suction conduit by the suction control apparatus, to generate a backflow, when it is detected that the flow rate of the fluid is equal to or lower than a specified value or the internal pressure of the suction conduit is equal to or higher than a specified value by the fluid detection apparatus and to cause the suction control apparatus to perform re-suction at a second flow velocity that is greater than the first flow velocity after continuing the backflow for a predetermined period of time.

Furthermore, a fluid perfusion method according to an aspect of the present invention includes: suctioning a fluid inside a living body at a first flow velocity via a suction conduit; after suctioning for a predetermined period of time, reversing a flow of a liquid that is suctioned via the suction conduit, to generate a backflow; performing re-suction at a second flow velocity that is higher than the first flow velocity after continuing the backflow; and performing suction at the first flow velocity after performing the re-suction for a predetermined period of time.

In addition, a fluid perfusion method according to another aspect of the present invention includes: suctioning a fluid inside a living body at a first flow velocity via a suction conduit; detecting a flow rate of the fluid that flows through the suction conduit or an internal pressure of the suction conduit; and reversing a flow of the fluid that is suctioned via the suction conduit, to generate a backflow, when it is detected that the flow rate of the fluid is equal to or lower than a specified value or the internal pressure of the suction conduit is equal to or higher than a specified value and performing re-suction at a second flow velocity that is greater than the first flow velocity after continuing the backflow for a predetermined period of time.

### BRIEF DESCRIPTION OF THE DRAWINGS

FIG. 1 is a schematic configuration diagram showing a medical system including a fluid perfusion apparatus according to a first embodiment of the present invention;

FIG. 2 is an explanatory diagram for describing a distal end portion of an endoscope insertion portion;

FIG. 3 is an explanatory diagram for describing the distal end portion of the endoscope insertion portion;

FIG. 4 is a diagram showing an example of processes of an occurrence of clogging in a suction conduit and clogging removal by a flow of water;

FIG. 5 is a block diagram showing a configuration of a fluid perfusion apparatus;

FIG. 6 is a diagram showing an example of a configuration of a drive mechanism 16;

FIG. 7 is a diagram showing an example of a relationship between a spring constant and a time it takes for a syringe to be restored when a preload of 2 mm is provided;

FIG. 8 is a diagram showing an example of a syringe connected to a branch midway along the suction conduit;

FIG. 9 is a schematic diagram of a case where the syringe is connected to a vicinity of an operating unit of an endoscope;

FIG. 10 is a schematic diagram of a case where the syringe is connected to a vicinity of the fluid perfusion apparatus;

FIG. 11 is a diagram showing a relationship between a flow rate of a backflow that satisfies both a clogging prevention effect and prevention of blasting of fragmented stones and a flow rate of suction by perfusion;

FIG. 12 is a flow chart for explaining an example of perfusion control by a fluid perfusion apparatus 10 according to the first embodiment;

FIG. 13 is a flow chart for explaining another example of the perfusion control by the fluid perfusion apparatus 10;

FIG. 14 is a flow chart for explaining an example of a more detailed control method of the perfusion control by the fluid perfusion apparatus 10;

FIG. 15 is a flow chart for explaining another example of a more detailed control method of the perfusion control by the fluid perfusion apparatus 10;

FIG. 16 is a flow chart for explaining another example of a more detailed control method of the perfusion control by the fluid perfusion apparatus 10;

FIG. 17 is a flow chart for explaining another example of a more detailed control method of the perfusion control by the fluid perfusion apparatus 10;

FIG. 18 is a diagram showing another example of a configuration of the drive mechanism 16;

FIG. 19 is a diagram showing another example of a configuration of the drive mechanism 16;

FIG. 20 is a diagram showing another example of a configuration of the drive mechanism 16;

FIG. 21 is a diagram showing another example of a configuration of the drive mechanism 16;

FIG. 22 is a diagram showing another example of a configuration of the drive mechanism 16;

FIG. 23 is a diagram showing another example of a configuration of the drive mechanism 16;

FIG. 24 is a diagram showing another example of a configuration of the drive mechanism 16;

FIG. 25 is a diagram showing another example of a configuration of the drive mechanism 16;

FIG. 26 is a diagram showing another example of a configuration of the drive mechanism 16;

FIG. 27 is a diagram showing another example of a configuration of the drive mechanism 16;

FIG. 28 is a diagram showing another example of a configuration of the drive mechanism 16;

FIG. 29 is a diagram showing another example of a configuration of the drive mechanism 16;

FIG. 30 is a diagram showing a relationship between a flow rate of a backflow that removes clogging and a flow rate of suction by perfusion; and

FIG. 31 is a flow chart for explaining an example of perfusion control by the fluid perfusion apparatus 10 according to a second embodiment.

### DETAILED DESCRIPTION

Generally, a perfusion state is detected based on a relationship among a drive force of a pump, a flow rate of a fluid that flows through a conduit, pressure in the conduit, and the like. In other words, when there is an abnormality in a perfusion state, snagging of a stone in a suction conduit has already started. Therefore, when detecting a perfusion state of a conduit and controlling a flow of a liquid in the conduit based on a result of detection of a perfusion state, a stone cannot be prevented from becoming snagged in the suction conduit.

According to the embodiments described below, a fluid perfusion apparatus and a fluid perfusion method capable of preventing a stone from becoming snagged in a suction conduit and, even when a stone becomes snagged in the suction conduit, capable of immediately removing the snagging can be provided.

Hereinafter, embodiments of the present invention will be described in detail with reference to the drawings.

### First Embodiment

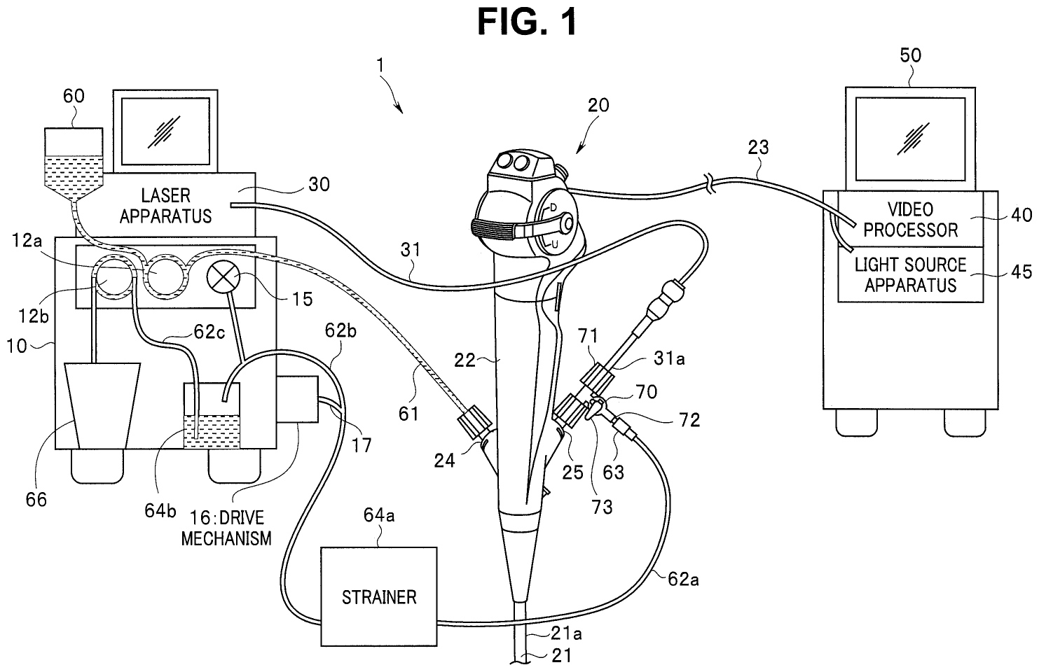

FIG. 1 is a schematic configuration diagram showing a medical system including a fluid perfusion apparatus according to a first embodiment of the present invention.

As shown in FIG. 1, a medical system 1 includes a fluid perfusion apparatus 10, an endoscope 20, a laser apparatus 30, a video processor 40, a light source apparatus 45, and a monitor 50. The endoscope 20 has an elongated insertion portion 21 and an operating unit 22. The insertion portion 21 of the endoscope 20 is inserted into an organ such as a kidney of a subject and the endoscope 20 is configured to pick up an image of the organ and output an image pickup signal.

In the insertion portion 21, for example, a flexible portion 21a is formed on a proximal end side, and a bending portion (not illustrated) is provided on a distal end side of the flexible portion 21a and a rigid distal end portion 26 (refer to FIG. 3) is provided on a distal end side of the bending portion in a consecutive manner. The operating unit 22 provided with various buttons for operating the endoscope 20 is arranged on a proximal end side of the insertion portion 21. The bending portion is configured to bend by operating the operating unit 22.

One end of a universal cord 23 is connected to the operating unit 22, and another end of the universal cord 23 is connected to the video processor 40 and the light source apparatus 45. The endoscope 20, the video processor 40, and the light source apparatus 45 are connected to each other through the universal cord 23 and various signals and illuminating light are transmitted.

The video processor 40 is configured to control an entirety of the medical system 1. The image pickup signal is inputted to the video processor 40 from the endoscope 20 via the universal cord 23, and the video processor 40 is configured to obtain an image signal by subjecting the inputted image pickup signal to signal processing. The video processor 40 is configured to output the image signal to the monitor 50. The monitor 50 is configured to display an image based on the image signal outputted by the processor 40.

For example, the light source apparatus 45 has a white LED and is configured to emit illuminating light. The illuminating light emitted by the light source apparatus 45 is guided to the rigid distal end portion 26 via the universal cord 23 and a light guide (not illustrated) inserted into the insertion portion 21.

The operating unit 22 is provided with a water feed tube mounting pipe sleeve 24 and a T-tube mounting pipe sleeve 25. A water feed tube 61 connected to a tank 60 is connected to the water feed tube mounting pipe sleeve 24. The water feed tube 61 is inserted until reaching a distal end of the rigid distal end portion 26 inside the insertion portion 21.

In addition, the operating unit 22 has an opening communicated with a suction channel 27 (refer to FIG. 3) provided inside the insertion portion 21 and the T-tube mounting pipe sleeve 25 is provided in the opening. A T-tube 70 is mounted to the T-tube mounting pipe sleeve 25. A laser fiber mounting opening 71 is provided in the T-tube 70. A fiber mounting portion 31a of a laser fiber 31 connected to the laser apparatus 30 is mounted to the laser fiber mounting opening 71. The laser fiber 31 can be inserted into the suction channel 27 via the T-tube 70 and the T-tube mounting pipe sleeve 25.

In addition, a drain pipe sleeve 72 is provided in the T-tube 70. A tube mounting portion 63 of a suction tube 62a is mounted to the drain pipe sleeve 72. The T-tube 70 is provided with a cock 73 configured to allow water suctioned from the suction channel 27 to flow to a side of the suction tube 62a but to prevent the water from flowing to a side of the laser fiber mounting opening 71.

The suction tube 62a is connected to a secondary strainer 64b via a primary strainer 64a and a suction tube 62b. The secondary strainer 64b is connected to a drain tank 66 via a suction tube 62c. Note that the suction tubes 62a, 62b, and 62c may also be referred to without any distinction as a suction tube 62. In addition, the primary strainer 64a and the secondary strainer 64b may be omitted and the suction tube 62a, the suction tube 62b, and the suction tube 62c may be coupled to each other.

The fluid perfusion apparatus 10 is provided with a water feed pump 12a that is a water feed source and a suction pump 12b that is a suction source. The water feed pump 12a and the suction pump 12b may be constituted of, for example, a tube pump. The water feed pump 12a is configured to supply a liquid that fills the tank 60 to organs inside the body via the water feed tube 61. In addition, the suction pump 12b is connected to the suction tube 62a via the suction tube 62c, the secondary strainer 64b, the suction tube 62b, and the primary strainer 64a and negative pressure of the suction tube 62c created by the suction pump 12b is transmitted to the suction tube 62a. In other words, a liquid suctioned from an organ inside the body by the suction pump 12b is discharged to the drain tank 66 via the suction channel 27, the suction tube 62a, the primary strainer 64a, the suction tube 62b, the secondary strainer 64b, and the suction tube 62c. The primary strainer 64a and the secondary strainer 64b may also be referred to without any distinction as a strainer 64.

In addition, the fluid perfusion apparatus 10 includes a drive mechanism 16. A detailed configuration of the drive mechanism 16 will be provided later. The drive mechanism 16 is connected to the suction tube 62b by a tube 17. While the tube 17 is connected to the suction tube 62b, the tube 17 is not limited thereto. For example, the tube 17 may be configured to be connected to the suction tube 62a in a vicinity of the operating unit 22.

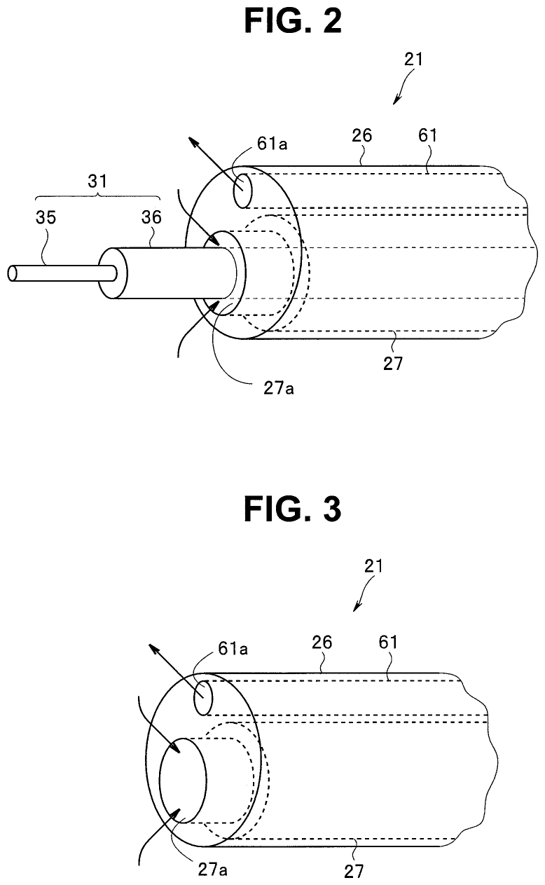

FIG. 2 and FIG. 3 are explanatory diagrams for describing a distal end portion of an endoscope insertion portion.

An illumination window (not illustrated) which a distal end surface of the light guide faces and an observation window (not illustrated) for guiding a subject optical image to a light receiving surface of an image pickup device (not illustrated) are arranged on a distal end surface of the rigid distal end portion 26 of the insertion portion 21. In the present embodiment, a distal end opening 61a of the water feed tube 61 is arranged on the distal end surface of the rigid distal end portion 26. An arrow shown in the distal end opening 61a in FIG. 2 and FIG. 3 indicates that a liquid is discharged from the distal end opening 61a of the water feed tube 61. By the water feed pump 12a, the liquid stored in the tank 60 (normal saline) is fed to an organ inside the body from the distal end surface of the rigid distal end portion 26 via the water feed tube 61 inserted into the insertion portion 21.

In addition, a distal end opening 27a of the suction channel 27 is arranged on the distal end surface of the rigid distal end portion 26. An arrow shown in the distal end opening 27a in FIG. 2 and FIG. 3 indicates that a liquid inside an organ inside the body is suctioned by the suction channel 27. By the suction pump 12b, the liquid inside an organ of the body is discharged to the drain tank 66 via the suction channel 27, the suction tube 62a, the primary strainer 64a, the suction tube 62b, the secondary strainer 64b, and the suction tube 62c.

While an example in which the suction channel 27 and the suction tube 62 are used as a suction conduit is shown in the present embodiment, by inserting a suction tube into the suction channel 27 and extending the suction tube to the outside via the T-tube 70, the suction tube may be used as a suction conduit to drain water from the organ to the outside.

In the example shown in FIG. 2, the laser fiber 31 inserted from the T-tube 70 is inserted into the suction channel 27 and arranged inside the suction channel 27 in a state where a distal end protrudes from the distal end surface of the rigid distal end portion 26. The laser fiber 31 is constituted of a core/clad 35 and a jacket 36 which covers the core/clad 35. The laser apparatus 30 is configured to emit, via the laser fiber 31, laser light from the distal end of the laser fiber 31.

During collection of stones, as shown in FIG. 2, the laser fiber 31 is inserted into the suction channel 27 and an endoscopic image of the inside of an organ is obtained by the endoscope 20 in a state where a distal end of the laser fiber 31 protrudes from the distal end opening 27a. In other words, illuminating light guided by the light guide (not illustrated) illuminates a subject from the illumination window (not illustrated) of the distal end surface of the rigid distal end portion 26. Reflected light from the subject passes through the observation window (not illustrated) and is received by an image pickup device. The image pickup device is configured to acquire an image pickup signal based on a subject optical image and to output the image pickup signal to the video processor 40 via a cable (not illustrated) inside the insertion portion 21 and the universal cord 23. The video processor 40 is configured to display an endoscopic image based on the image pickup signal on the monitor 50. Accordingly, an operator can observe, on the monitor 50, a state inside an organ in which the rigid distal end portion 26 is arranged. The operator points the distal end of the laser fiber 31 toward a stone inside the organ and operates the laser apparatus 30 to irradiate the stone with a laser while viewing the endoscopic image. The stone irradiated with the laser is fragmented and becomes relatively small fragments.

In the present embodiment, in the state shown in FIG. 2, a liquid is drained from inside the organ while feeding water into the organ by actions of the water feed pump 12a and the suction pump 12b. By this perfusion action, the stone inside the organ is suctioned into the suction channel 27 from a gap between the laser fiber 31 inserted into the suction channel 27 and an inner surface of the suction channel 27 and is discharged to the suction tube 62a via the T-tube 70.

Once laser irradiation by the laser fiber 31 ends, the laser fiber 31 is extracted from the laser fiber mounting opening 71. Accordingly, as shown in FIG. 3, the laser fiber 31 is removed from the suction channel 27. Thereafter, the stone is discharged to the outside of the body via the suction channel 27 that is relatively wide. While the laser fiber 31 is inserted into the suction channel 27 in FIG. 2 and FIG. 3, the laser fiber 31 is not limited thereto. For example, a configuration may be adopted in which the insertion portion 21 is provided with a channel to allow insertion of the laser fiber separately from the suction channel 27 and the laser fiber 31 is inserted via the channel to allow insertion of the laser fiber.

Since collection of a stone is performed in a state where the laser fiber 31 is inserted into the suction channel 27, the stone is to pass through a relatively narrow drain path between the laser fiber 31 and the inner surface of the suction channel 27 and the stone is likely to become snagged between the suction channel 27 and the laser fiber 31. In addition, the suction channel 27 is a relatively narrow drain path and, even when suction is performed in a state where the laser fiber 31 has been removed from the suction channel 27 as in FIG. 3, the stone may become snagged in the suction channel 27. Once the stone becomes snagged, the snagged stone acts as a starting point of an occurrence of clogging in which subsequent stones become snagged and which may eventually lead to obstruction of the suction channel 27. For example, when such an obstruction of the suction channel 27 occurs during collection of a stone inside the kidney, a rise in intrarenal pressure may become a concern.

In consideration thereof, the present embodiment enables snagging of a stone in the suction conduit to be prevented and, even when a stone becomes snagged in the suction conduit, enables the snagging to be immediately removed.

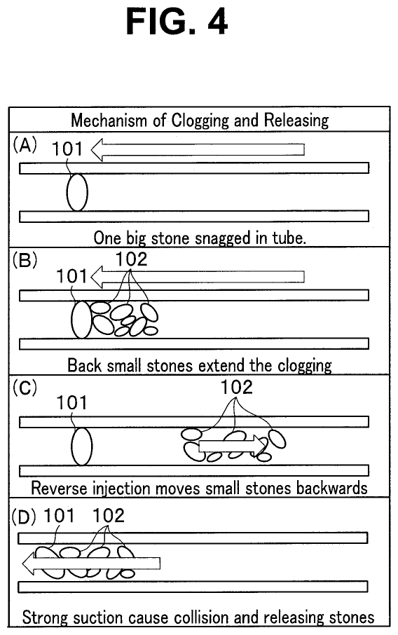

FIG. 4 is a diagram showing an example of processes of an occurrence of clogging in a suction conduit and the clogging removal by a flow of water.

First, as shown in (A) in FIG. 4, an occurrence of clogging is initiated by a single large fragmented stone 101 becoming snagged and immobile inside suction conduits constituted of the suction channel 27 and the suction tube 62. When a ratio between a diameter of the distal end opening 27a and a diameter of the suction channel 27 described above is sufficiently large, even if the fragmented stone to be suctioned is large, the stone should be smaller than an inner diameter of the suction channel 27. However, when the fragmented stone has a prominently elongated shape, a phenomenon may occur where the fragmented stone passes through the distal end opening 27a due to a minor radius being small in a longitudinal orientation along the conduit but changes to a lateral orientation once inside the suction channel 27 and becomes snagged inside the suction channel 27 due to a major radius being large. In fact, an experiment has shown that a frequency of occurrence of clogging rises when a large number of elongated fragmented stones are suctioned.

Next, as shown in (B) in FIG. 4, subsequent fragmented stones 102 line up behind the single snagged fragmented stone 101. Accordingly, the clogging grows and, as a degree of blockage of the suction channel 27 increases, an impact on a flow rate and pressure also increases. This concludes the description of a mechanism of occurrence of clogging.

Thereafter, the diagram represents a mechanism of removal of clogging. (C) in FIG. 4 represents a state where the subsequent fragmented stones 102 that had lined up in (B) in FIG. 4 are caused to retreat by a backflow. At this point, the single large fragmented stone 101 having caused the clogging often remains snagged and immobile. Since the subsequent fragmented stones 102 are only blocked from advancing by the large fragmented stone 101 and are not fixed with respect to the suction channel 27, the fragmented stones 102 are able to move in an opposite direction by creating a reverse flow.

(D) in FIG. 4 represents a state where the retreated fragmented stones 102 advance once again and collide with the fragmented stone 101 having caused the clogging, remove the snagging of the fragmented stone 101 having caused the clogging by impact, and continue flowing. It is difficult to generate an amount of force required to remove the snagged fragmented stone 101 by a flow of water alone. In fact, an observation of an experiment has revealed that clogging is removed by a backflow not when a flow in an opposite direction is being generated but when a flow of suction is subsequently generated and the fragmented stones collide with each other. In other words, generating a small backflow at a location where clogging has occurred and, subsequently, immediately generating a strong suction is effective in removing the clogging.

When performing a clogging removal operation by a backflow and suction as described above, a method of “regularly performing the operation before an occurrence or a development of clogging is detected” and a method of “performing the operation when an occurrence or a development of clogging is detected until the clogging is removed” are respectively conceivable. In the present embodiment, the method of “regularly performing the operation before an occurrence or a development of clogging is detected” will be described.

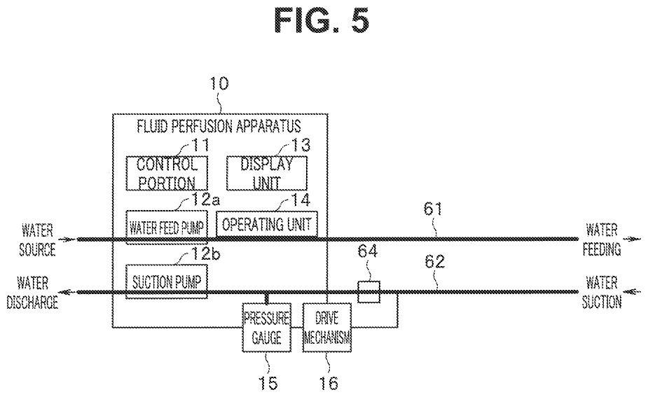

FIG. 5 is a block diagram showing a configuration of a fluid perfusion apparatus.

In FIG. 5, the fluid perfusion apparatus 10 as a medical apparatus includes a control circuit 11, the water feed pump 12a, the suction pump 12b, a display unit 13, an operating unit 14, a pressure gauge 15, and the drive mechanism 16. In FIG. 5, the primary strainer 64a and the secondary strainer 64b in FIG. 1 are shown as a strainer 64 and the suction tubes 62a, 62b, and 62c are shown as the suction tube 62.

The control circuit 11 may be constituted of a processor using a CPU (central processing unit), an FPGA (field programmable gate array), or the like. The control circuit 11 may be configured to operate according to a program stored in a memory (not illustrated) or a part or all of functions of the control circuit 11 may be realized by an electronic circuit of hardware. Note that the control circuit 11 may be constituted of a single processor or a plurality of processors.

The control circuit 11 is configured to control each unit of the fluid perfusion apparatus 10. The control circuit 11 is configured to generate a drive output for driving the water feed pump 12a and output the drive output to the water feed pump 12a. In addition, the control circuit 11 is configured to generate a drive output for driving the suction pump 12b and output the drive output to the suction pump 12b. By operating based on the drive output, the suction pump 12b is configured to generate predetermined suction pressure inside the suction conduits constituted of the suction channel 27 and the suction tube 62. For example, when it is assumed that a conduit resistance of the suction conduits constituted of the suction channel 27 and the suction tube 62 (hereinafter, the suction conduits will be simply referred to as a suction conduit) is constant, the suction pump 12b can cause a liquid with a flow rate approximately proportional to the drive output to flow through the suction conduit. In other words, in this case, the flow rate of the suction conduit increases or decreases in proportion to the drive output.

The pressure gauge 15 is provided midway along the suction conduit constituted of the suction tube 62 from the strainer 64 to the suction pump 12b. The pressure gauge 15 that constitutes a fluid detection apparatus is configured to measure pressure inside the suction conduit and output a measurement result to the control circuit 11.

The fluid perfusion apparatus 10 may include a flow meter configured to measure a flow rate of a fluid that flows through the suction flow path. However, for example, when a correlation between a rotating speed of a motor configured to operate the suction pump 12b and the flow rate of the suction conduit is known, a flow meter need not be provided since the flow rate of the suction conduit can be estimated.

Based on a measurement result of the pressure gauge 15 (or a measurement result of the flow meter), the control circuit 11 is configured to detect clogging inside the suction conduit and to drive the drive mechanism 16 or to control the water feed pump 12a and the suction pump 12b. When the control circuit 11 detects that an abnormality (clogging) has occurred in the suction conduit based on the measurement result of the pressure gauge 15, the control circuit 11 is configured to output warning information indicating that a possibility of obstruction of the suction conduit has arisen to the display unit 13. The display unit 13 is configured to display a warning based on the warning information from the control circuit 11. In this manner, the control circuit 11 constitutes an abnormality notification system configured to, when an abnormality is detected, notify the abnormality.

A user such as an operator can use the operating unit 14 to set a flow rate of a flow through the suction conduit. The operating unit 14 is configured to output setting information set by the user to the control circuit 11. The control circuit 11 is configured to control each unit of the fluid perfusion apparatus 10 such as the suction pump 12b based on the setting information from the operating unit 14.

As will be described later, the drive mechanism 16 is constituted of, for example, a combination of a syringe and a cam or a combination of a syringe and a linear actuator. A backflow and suction are generated in the suction conduit by operating the cam or the linear actuator based on control by the control circuit 11 to push in and pull the syringe.

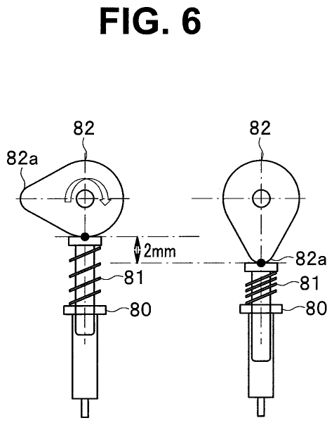

Next, a configuration of the drive mechanism 16 configured to generate a backflow and suction in the suction conduit will be described. FIG. 6 is a diagram showing an example of a configuration of the drive mechanism 16.

As shown in FIG. 6, the drive mechanism 16 that constitutes a suction control apparatus is constituted of a syringe 80 and a cam 82. The syringe 80 includes a spring 81 to be a restoring force. The cam 82 includes a projecting portion 82a for pushing the syringe 80 inward.

By causing the cam 82 to rotationally move under the control of the control circuit 11, the projecting portion 82a pushes the syringe 80 inward. Accordingly, a backflow is generated in the suction conduit. Subsequently, the syringe 80 is pulled by a restoring force of the spring 81. Accordingly, suction is generated in the suction conduit.

In order to remove clogging, as described earlier, generating a small backflow at a location where the clogging has occurred and, subsequently, immediately generating a strong suction is effective. In the present embodiment, the syringe 80 is connected midway along the suction conduit, a backflow is generated by pushing the syringe 80 inward, and a strong suction is generated by pulling the syringe 80.

However, when the syringe 80 does not include the spring 81, it is difficult to immediately perform the pulling operation after pushing and, in addition, a delay in the start of pulling may give rise to a possibility that removal of clogging will not be performed in a sufficient manner. In addition, when the syringe 80 does not include the spring 81, since high negative pressure created by the suction pump 12b is applied inside the suction conduit, water pooled inside the syringe 80 is suctioned and causes the syringe 80 to be pushed naturally, which creates the hassle of having the syringe 80 suction the water first before performing water feeding for removal of the clogging.

In contrast, when the syringe 80 includes the spring 81 as in the present embodiment, the syringe 80 is able to maintain a state where a predetermined amount of water is present inside the syringe and, by pushing the syringe 80 with the cam 82, a strong suction can be naturally generated inside the suction channel 27 in the process of the syringe 80 returning to its original position.

In addition, when pushing and pulling the syringe 80, the spring 81 may be provided with a preload so that, even in a most pulled state of the syringe 80, the spring 81 contracts by a maximum of around a few mm. Accordingly, a period of time it takes to pull the syringe 80 can be reduced while reducing an amount of force necessary for pushing the syringe 80 with the spring 81 with a smaller spring constant. Reducing the period of time it takes to pull the syringe 80 has an effect of increasing a momentum of the suction inside the suction channel 27 which is generated during the pull and, since an impact between the fragmented stones in the clogging removal mechanism described above can be increased, a clogging removal effect can be further enhanced.

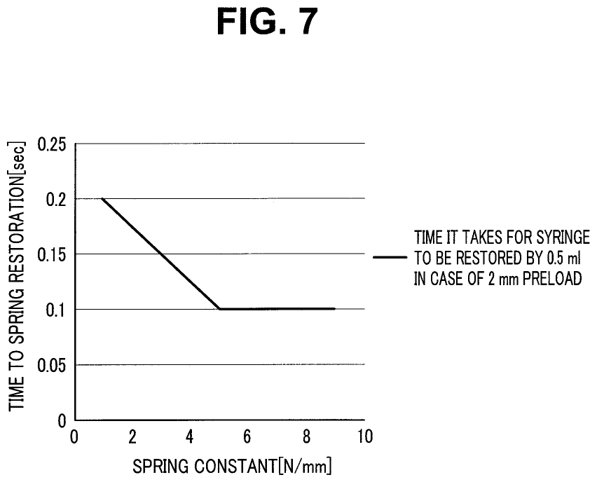

FIG. 7 is a diagram showing an example of a relationship between a spring constant and a time it takes for a syringe to be restored when a preload of 2 mm is provided.

In order to pull the syringe 80 and automatically apply suction against suction pressure inside the suction channel 27, the spring 81 with a spring constant of 1 N/mm or more and 5 N/mm or less is desirably used. In particularly, as shown in FIG. 7, a restoration time of the spring 81 can be minimized with a smallest amount of force when the spring constant is 5 N/mm.

In addition, as shown in FIG. 7, when a preload of around 2 mm is provided, setting the spring constant larger than 5 N/mm simply increases an amount of force necessary for pushing the spring 81 while the period of time required to pull the syringe 80 is more or less the same. Therefore, the restoration time of the spring 81 can be minimized by setting the spring constant to 5 N/mm.

In addition, a length of the spring 81 may be adjusted so that an amount of water inside the syringe 80 is around, for example, 1 ml in the most pulled state of the syringe 80. A water feeding amount by pushing the syringe 80 may be a small amount and, more specifically, clogging can be removed in an efficient manner without increasing intrarenal pressure when a water feeding amount ranges from around 0.1 to 1.5 ml.

In doing so, for example, when a content of the syringe 80 is 1 ml in a fully extended state of the spring 81, water feeding and suction of a constant amount of 1 ml can be repeated by a simple operation involving pushing the syringe 80 inward as far as possible and waiting for the syringe 80 to automatically return to its original position without having to adjust amounts of water feeding and suction of the syringe 80.

Clogging removal by pushing and pulling the syringe 80 as described above may conceivably be performed by both a system configured to perform the clogging removal when detecting an occurrence of the clogging based on a sensor value such as a flow rate or pressure in the perfusion as described above and a system configured to constantly repeat pushing and pulling during the perfusion. When performing removal after clogging is detected, since the clogging may not be removed by only pushing and pulling the syringe once, desirably, the syringe can be repetitively pushed and pulled a plurality of times such as 10 to 15 times. In doing so, by performing water feeding and suction by the suction pump 12b without stopping perfusion, a high clogging removal effect can be generated by a combination of the suction by the suction pump 12b and the suction by the syringe 80 while preventing a procedure time from being affected. The syringe 80 configured as described above is connected to a branch midway along the suction conduit.

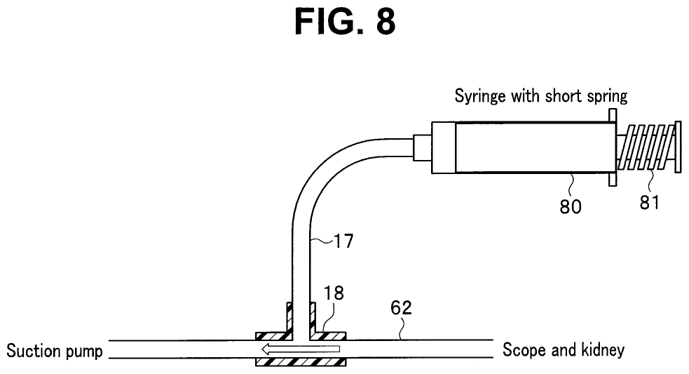

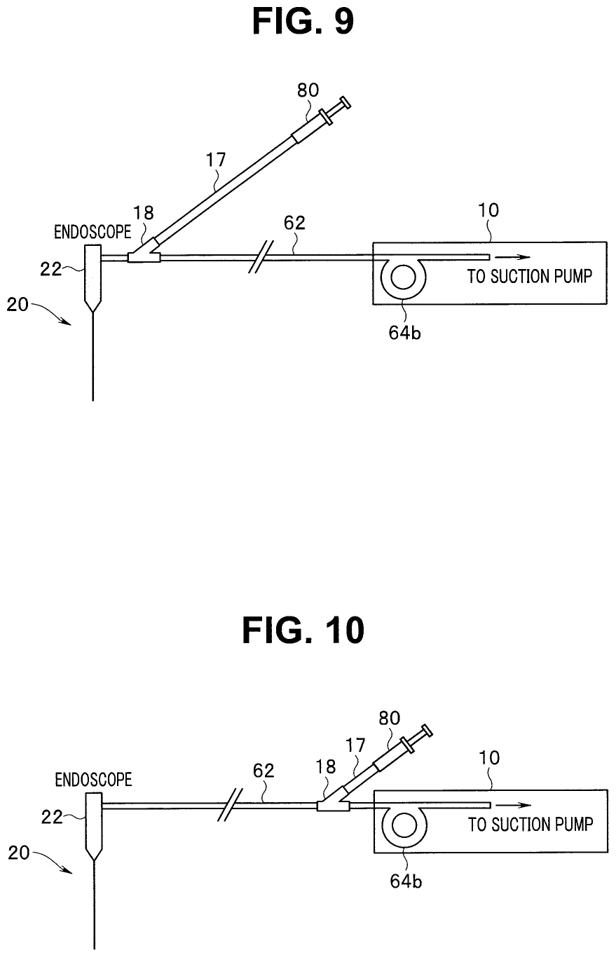

FIG. 8 is a diagram showing an example of a syringe connected to a branch midway along the suction conduit. FIG. 9 is a schematic diagram of a case where the syringe is connected to a vicinity of an operating unit of an endoscope. FIG. 10 is a schematic diagram of a case where the syringe is connected to a vicinity of a fluid perfusion apparatus.

As shown in FIG. 8, a branch 18 is provided midway along the suction conduit (the suction tube 62) from the endoscope 20 to the suction pump 12b and the syringe 80 is connected to the tube 17 that is connected to the branch 18.

A position of the branch 18 may be arranged in a periphery of the operating unit 22 of the endoscope 20 as shown in FIG. 9. Alternatively, the position of the branch 18 may be arranged in a periphery of the fluid perfusion apparatus 10 including the secondary strainer 64b as shown in FIG. 10.

When the branch 18 is arranged in a periphery of the operating unit 22 of the endoscope 20, since a conduit from the branch 18 that connects the syringe 80 to the suction channel 27 where clogging has occurred is short, there is an advantage in that a clogging removal effect by pushing and pulling the syringe increases (a retreat amount of the subsequent fragmented stones 102 and the impact of collision with the leading fragmented stone 101 described above increase).

However, when the branch 18 is arranged in the periphery of the operating unit 22 of the endoscope 20, the syringe 80 may get in the way of an operator of the endoscope. In addition, when the pushing and pulling operation of the syringe 80 is entrusted to a helper, since the helper must closely approach the endoscope operator, a problem arises in that it is difficult to perform the operation. Therefore, a configuration is desirable in which the tube 17 from the branch 18 to which the syringe 80 is connected to the syringe 80 is made longer, the branch 18 is provided on the operating unit 22 of the endoscope 20, and the syringe 80 and the cam 82 for pushing the syringe 80 are provided in a periphery of the fluid perfusion apparatus 10.

Furthermore, when a fragmented stone collected from the suction channel 27 flows into the tube 17 connecting the syringe 80 from the branch 18, the fragmented stone may become clogged inside the tube 17 and obstruct a flow created by pushing and pulling the syringe 80. Therefore, an angle of the branch 18 is desirably provided so that the tube 17 to which the syringe 80 is connected is oriented in a direction opposite to gravity from the branch 18 in order to prevent fragmented stones from flowing into the tube 17.

On the other hand, when the branch 18 is provided in a periphery of the fluid perfusion apparatus 10 at, for example, midway along a suction conduit between the primary strainer 64a and the secondary strainer 64b, a clogging removal effect is reduced due to a longer length of the conduit to the clogging inside a suction channel 27b. However, clogging up to around a few cm can be sufficiently removed and, for example, by fixing the syringe 80 to the fluid perfusion apparatus 10, there is an advantage that the pushing and pulling operation of the syringe 80 can be readily automated by the cam 82 or the like.

A difference in clogging removal effects due to positions of the branch 18 to which the syringe 80 is connected via the tube 17 is created due to a magnitude of respective conduit resistances from the branch 18 to the side of the endoscope 20 and from the branch 18 to the side of the suction pump 12b. In other words, while a liquid fed from the syringe 80 flows in a divided manner to both the side of the endoscope 20 and the side of the suction pump 12b from the branch 18, a larger amount of the liquid is to flow toward the side with the lower conduit resistance. Therefore, since the closer the position of the branch 18 to the endoscope 20, the lower the conduit resistance on the side of the endoscope 20, a larger amount of the liquid is to flow into the suction channel 27b with the clogging to be removed. As a result, by arranging the branch 18 in the vicinity of the operating unit 22 of the endoscope 20, a strong backflow and suction can be applied with a same water feeding amount of the syringe 80.

In the present embodiment, the drive mechanism 16 including the syringe 80 and the cam 82 as described above is driven to continuously generate a backflow and suction before clogging occurs. When continuously generating a backflow, since laser fragmentation and collection of stones are to be continuously performed even when a backflow is being repetitively generated, an excessively strong backflow ends up causing water to spout into a kidney from the distal end of the suction channel and causing the stone or fragments inside the kidney to be blasted away by a flow of water. Accordingly, by moving away fragments to be irradiated with a laser from a desired portion, there is a risk of impeding a procedure and prolonging a procedure time.

On the other hand, when a backflow is too weak, there may be cases where occurrences of clogging cannot be reduced or, when clogging occurs, the clogging cannot be removed. Therefore, a flow rate of a backflow that satisfies both a clogging prevention effect and prevention of blasting of fragmented stones must be set.

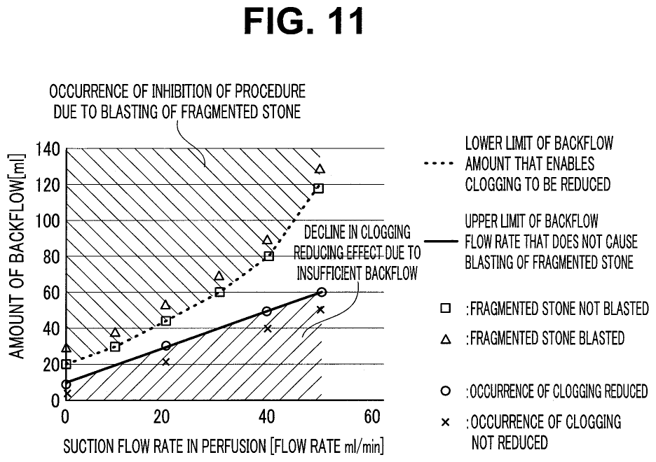

FIG. 11 is a diagram showing a relationship between a flow rate of a backflow that satisfies both a clogging prevention effect and prevention of blasting of fragmented stones and a flow rate of suction by perfusion.

As shown in FIG. 11, when the flow rate of suction by perfusion (a perfusion amount of the endoscope 20) is set to 10 to 50 ml/min, an upper limit value of the flow rate of a backflow must be kept to or lower than around 20 to 120 ml/min. On the other hand, a lower limit value of the flow rate of the backflow must be set to 10 to 60 ml/min or higher as a flow rate necessary for reducing clogging.

While the flow rate of the backflow must be set between the upper limit value and the lower limit value, since it is known that the higher the flow rate, the greater the clogging removal effect, conceivably, the flow rate of the backflow is optimally set to as large a value within the permissible range as possible or, in other words, set to the upper limit value of the flow rate of the backflow. For example, when the flow rate of suction by perfusion is 30 ml/min, the flow rate of the backflow is favorably around 60 ml/min.

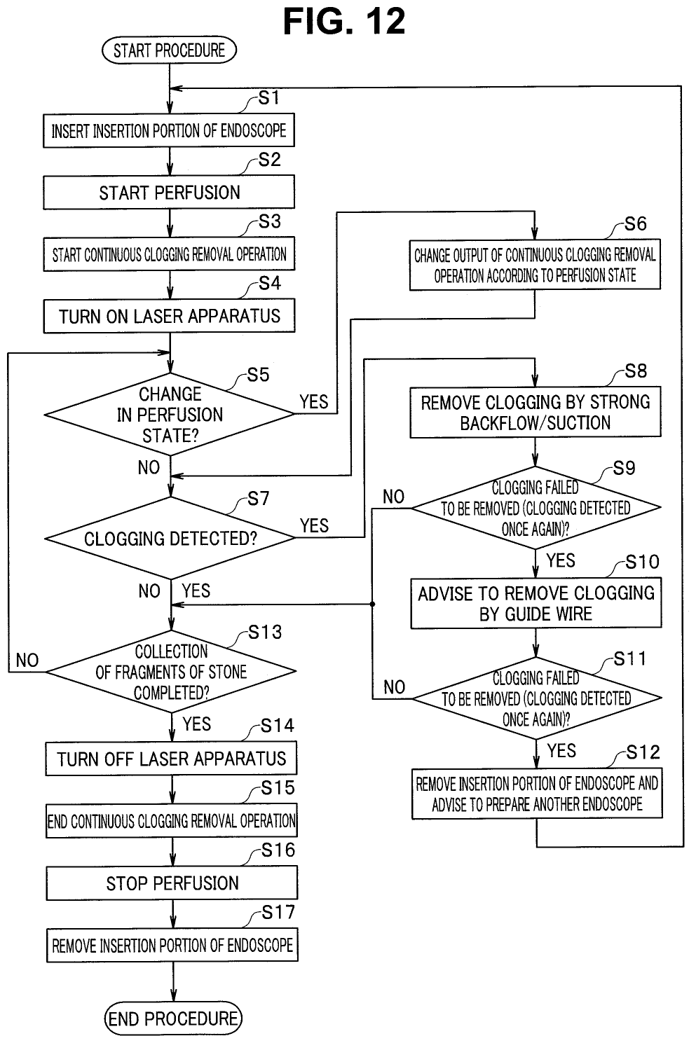

Next, operations of the embodiment configured as described above will be explained with reference to FIG. 12. FIG. 12 is a flow chart for explaining an example of perfusion control by the fluid perfusion apparatus 10 according to the first embodiment.

First, when a procedure is started, the insertion portion 21 of the endoscope 20 is inserted into an organ of a subject (S1). The control circuit 11 controls the water feed pump 12a and the suction pump 12b and starts perfusion (S2).

Next, the control circuit 11 starts a continuous clogging removal operation (S3). More specifically, the control circuit 11 drives the drive mechanism 16 and rotationally moves the cam 82. Accordingly, pushing and pulling of the syringe 80 are continuously performed to perform the continuous clogging removal operation involving a backflow and suction.

Next, the laser apparatus 30 is turned on (S4). Then, the distal end of the laser fiber 31 is pointed toward a stone inside the organ and the stone is irradiated with a laser. By irradiating the stone with a laser, the stone is fragmented and becomes fragmented stones to be collected via the suction channel 27b.

Next, the control circuit 11 determines whether or not a perfusion state has changed (S5). More specifically, the control circuit 11 determines whether or not a perfusion state has changed based on a measurement result of the pressure gauge 15.

When the control circuit 11 determines that the perfusion state has changed (S5: YES), the control circuit 11 changes an output of the continuous clogging removal operation in accordance with the perfusion state (S6) and advances to processing of step S7. In other words, when a suction flow rate in the perfusion changes, the control circuit 11 suitably changes a flow rate of a backflow in accordance with the suction flow rate in the perfusion.

On the other hand, when the control circuit 11 determines that the perfusion state has not changed in the processing of step S5 (S5: NO) or when the control circuit 11 performs the processing of step S6, the control circuit 11 determines whether or not clogging has been detected (S7). The control circuit 11 detects clogging of the suction conduit based on a measurement result of the pressure gauge 15. When the control circuit 11 detects clogging, the control circuit 11 advances to step S8 and performs processing of clogging removal.

In the processing of clogging removal, first, the control circuit 11 performs clogging removal by a strong backflow and suction (S8). The control circuit 11 generates a strong backflow and suction by, for example, increasing an output of the continuous clogging removal operation of step S3.

Next, the control circuit 11 determines whether or not the clogging could not be removed (S9). When the control circuit 11 determines that the clogging could be removed (S9: NO), the control circuit 11 advances to processing of step S13. On the other hand, when the control circuit 11 determines that the clogging failed to be removed (S9: YES), the control circuit 11 advises to remove the clogging with a guide wire (S10). Note that a clogging removal tool is not limited to a guide wire and another dedicated clogging removal tool may be prepared.

Next, the control circuit 11 determines whether or not the clogging could not be removed (S11). When the control circuit 11 determines that the clogging could be removed (S11: NO), the control circuit 11 advances to the processing of step S13. On the other hand, when the control circuit 11 determines that the clogging could not be removed (S11: YES), the control circuit 11 advises to extract the insertion portion 21 of the endoscope 20 and prepare another endoscope 20 (S12), and returns to step S1.

When the control circuit 11 determines that clogging was not detected in the processing of step S7 (S7: NO) or when the control circuit 11 determines that the clogging could be removed in the processing of step S9 or S11, the control circuit 11 determines whether or not collection of fragments of the stone has been completed (S13).

When the control circuit 11 determines that collection of fragments of the stone has not been completed (S13: NO), the control circuit 11 returns to step S5 and repeats similar processing. On the other hand, when the control circuit 11 determines that collection of fragments of the stone has been completed (S13: YES), the control circuit 11 turns off the laser apparatus 30 (S14).

Next, the control circuit 11 stops the continuous clogging removal operation (S15). More specifically, the control circuit 11 stops driving of the drive mechanism 16 and stops continuous pushing and pulling of the syringe 80.

Next, the control circuit 11 controls the water feed pump 12a and the suction pump 12b and stops perfusion (S16). Finally, the insertion portion 21 of the endoscope 20 is extracted from the organ of the subject (S17) to finish the procedure.

As described above, when perfusion starts, the fluid perfusion apparatus 10 starts a continuous clogging removal operation. In other words, by driving the drive mechanism 16 made up of the syringe 80 and the cam 82 and constantly generating a backflow and suction in the suction conduit, the fluid perfusion apparatus 10 can prevent stones from becoming snagged in the suction conduit.

In addition, when the fluid perfusion apparatus 10 detects clogging of the suction conduit, the fluid perfusion apparatus 10 can immediately remove the clogging inside the suction conduit by generating a strong backflow and suction.

Therefore, the fluid perfusion apparatus 10 according to the present embodiment is capable of preventing a stone from becoming snagged in a suction conduit and, even when a stone becomes snagged in the suction conduit, the snagging can be immediately removed.

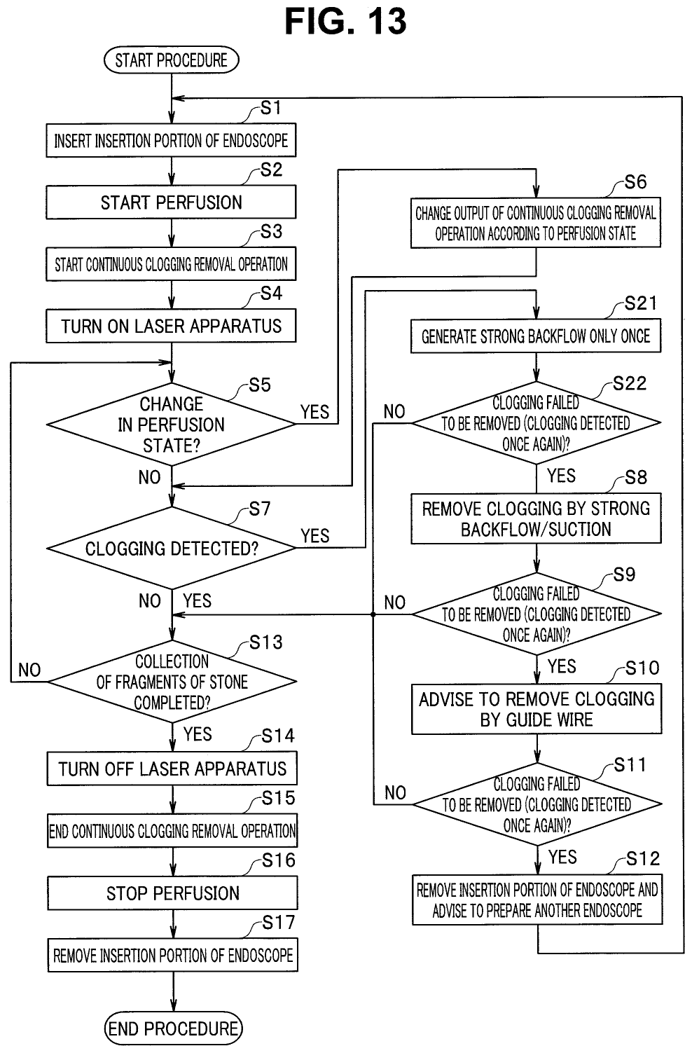

Note that the perfusion control by the fluid perfusion apparatus 10 is not limited to the processing shown in FIG. 12. FIG. 13 is a flow chart for explaining another example of the perfusion control by the fluid perfusion apparatus 10. In FIG. 13, similar processing to FIG. 12 will be denoted by same reference signs and a description of such processing will be omitted.

When the control circuit 11 determines that clogging was detected in the processing of step S7, the control circuit 11 generates a strong backflow only once (S21). As a specific embodiment for generating a strong backflow only once, there is a method of instantaneously opening the suction conduit to positive pressure using a solenoid valve or the like or, in the case of a configuration where the syringe 80 or the like is provided midway along the suction conduit as in the present embodiment, a method of temporarily increasing output of the clogging removal operation described above that is continuously repeated.

Subsequently, the control circuit 11 determines whether or not the clogging could not be removed (S22). When the control circuit 11 determines that the clogging failed to be removed (S22: YES), the control circuit 11 advances to the processing of step S8 and performs processing similar to that shown in FIG. 12. On the other hand, when the control circuit 11 determines that the clogging could be removed (S22: NO), the control circuit 11 advances to the processing of step S13 and performs processing similar to that shown in FIG. 12. Other processing steps are similar to those shown in FIG. 12.

In the present embodiment, as a method of detecting clogging, for example, a change in pressure inside the suction conduit is monitored by the pressure gauge 15. In this case, a temporary obstruction of the distal end opening 27a at a distal end of the suction conduit by a large fragmented stone or the like may possibly be also detected as clogging in addition to clogging of a fragmented stone inside the suction conduit.

When only the distal end opening 27a is obstructed in this manner, since it is highly likely that the obstruction can be removed by generating a strong backflow once without having to repetitively generate a strong backflow and suction in a similar manner to when removing clogging inside the suction conduit, a strong backflow is first generated only once when clogging is detected. By adopting a procedure in which clogging removal inside the suction conduit is performed anew only when the clogging has not been removed, the number of times the time-consuming clogging removal procedure is performed can be suppressed.

A more detailed control method of the perfusion control by the fluid perfusion apparatus 10 will now be described.

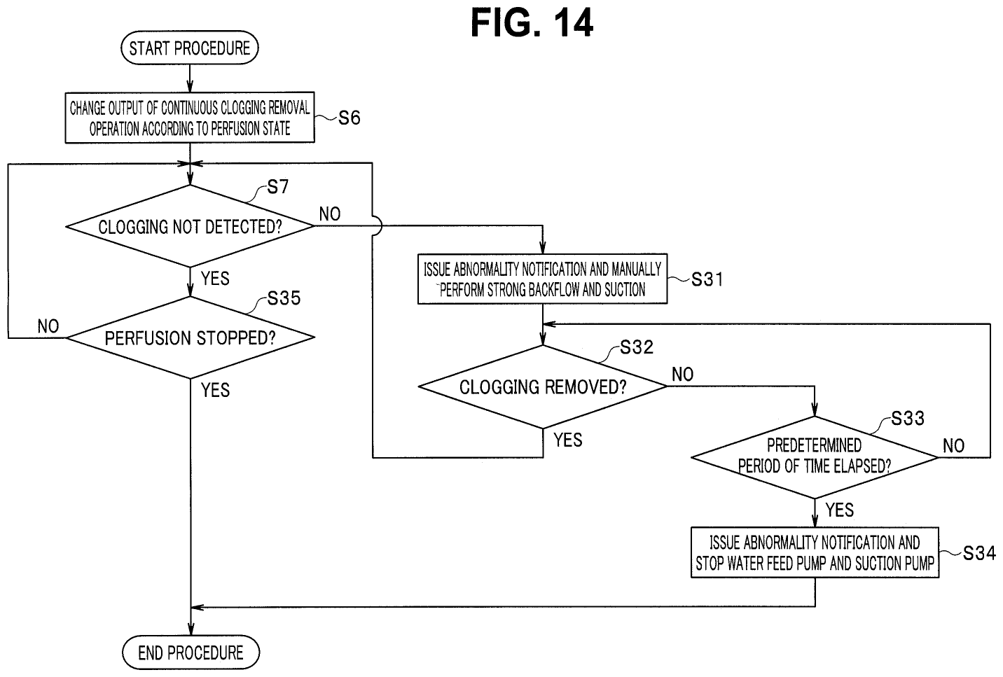

FIG. 14 is a flow chart for explaining an example of a more detailed control method of the perfusion control by the fluid perfusion apparatus 10. In FIG. 14, similar processing to FIG. 12 and FIG. 13 will be denoted by same reference signs and a description of such processing will be omitted.

First, when the control circuit 11 changes the output of the continuous clogging removal operation in accordance with the perfusion state in the processing of step S6 and detects clogging in the processing of step S7, the control circuit 11 issues an abnormality notification and advises to manually generate a strong backflow and suction (S31). The control circuit 11 causes the display unit 13 to display the abnormality notification. Note that the abnormality notification is not limited to being displayed on the display unit 13 and, for example, an abnormality may be notified using voice (sound), light, color, or the like.

In addition, the control circuit 11 may cause the number of times a strong backflow and suction need to be manually generated to be displayed on the display unit 13 based on a measurement result of the pressure gauge 15. Furthermore, the control circuit 11 may cause the number of times a strong backflow and suction need to be further manually generated to be displayed on the display unit 13 based on a measurement result of the pressure gauge 15. Moreover, the control circuit 11 may prompt a user to stop the water feed pump 12a and the suction pump 12b and to stop the laser apparatus 30 for the sake of safety.

Next, the control circuit 11 determines whether or not the clogging is removed (S32). When the control circuit 11 determines that the clogging is removed (S32: YES), the control circuit 11 returns to the processing of step S7. On the other hand, when the control circuit 11 determines that the clogging is not removed (S32: NO), the control circuit 11 determines whether or not a predetermined period of time has elapsed (S33).

When the control circuit 11 determines that the predetermined period of time has not elapsed (S33: NO), the control circuit 11 returns to the processing of step S32. On the other hand, when the control circuit 11 determines that the predetermined period of time has elapsed (S33: YES), the control circuit 11 issues an abnormality notification and stops the water feed pump 12a and the suction pump 12b (S34).

As described above, when clogging cannot be removed for a predetermined period of time even when a strong backflow and suction are manually generated, the control circuit 11 determines that severe clogging has occurred and issues an abnormality notification which differs from that in the processing of step S31. In addition, the control circuit 11 automatically stops the water feed pump 12a and the suction pump 12b and finishes the procedure.

On the other hand, when the control circuit 11 does not detect clogging in the processing of step S7, the control circuit 11 determines whether or not perfusion has been stopped (S35). When the control circuit 11 determines that the perfusion has not been stopped (S35: NO), the control circuit 11 returns to the processing of step S7. On the other hand, when the control circuit 11 determines that the perfusion has been stopped (S35: YES), the control circuit 11 finishes the procedure.

Note that a more detailed control method of the perfusion control by the fluid perfusion apparatus 10 is not limited to the processing shown in FIG. 14.

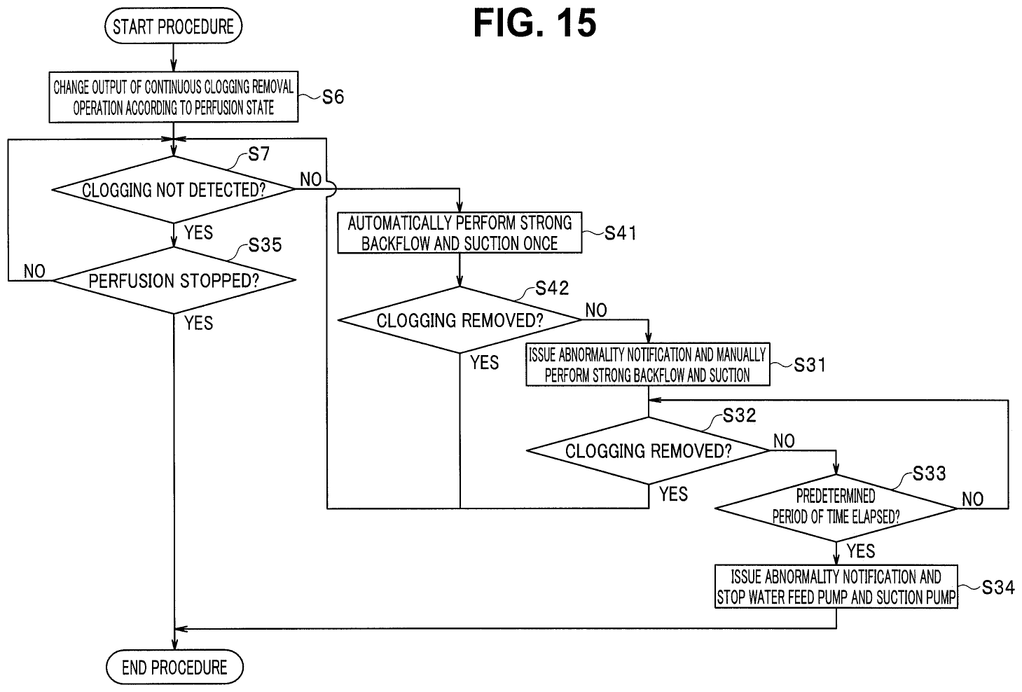

FIG. 15 is a flow chart for explaining another example of a more detailed control method of the perfusion control by the fluid perfusion apparatus 10. In FIG. 15, similar processing to FIG. 14 will be denoted by same reference signs and a description of such processing will be omitted.

When the control circuit 11 detects clogging in the processing of step S7, the control circuit 11 automatically generates a strong backflow and suction once (S41).

Next, the control circuit 11 determines whether or not the clogging is removed (S42). When the control circuit 11 determines that the clogging is removed (S42: YES), the control circuit 11 returns to the processing of step S7. On the other hand, when the control circuit 11 determines that the clogging is not removed (S42: NO), the control circuit 11 makes a transition to the processing of step S31. Processing of step S31 and thereafter are similar to those shown in FIG. 14.

As described above, control may be performed so that, after detecting clogging of the suction conduit, the drive mechanism 16 generates a stronger backflow and suction once. This is control in order to deal with a phenomenon which cannot be removed by a normal operation of the drive mechanism 16 but which can be removed by a strong backflow and suction. Accordingly, a frequency of aborting a procedure can be reduced, a surgical time can be shortened, and stress on an operator or the like can be alleviated.

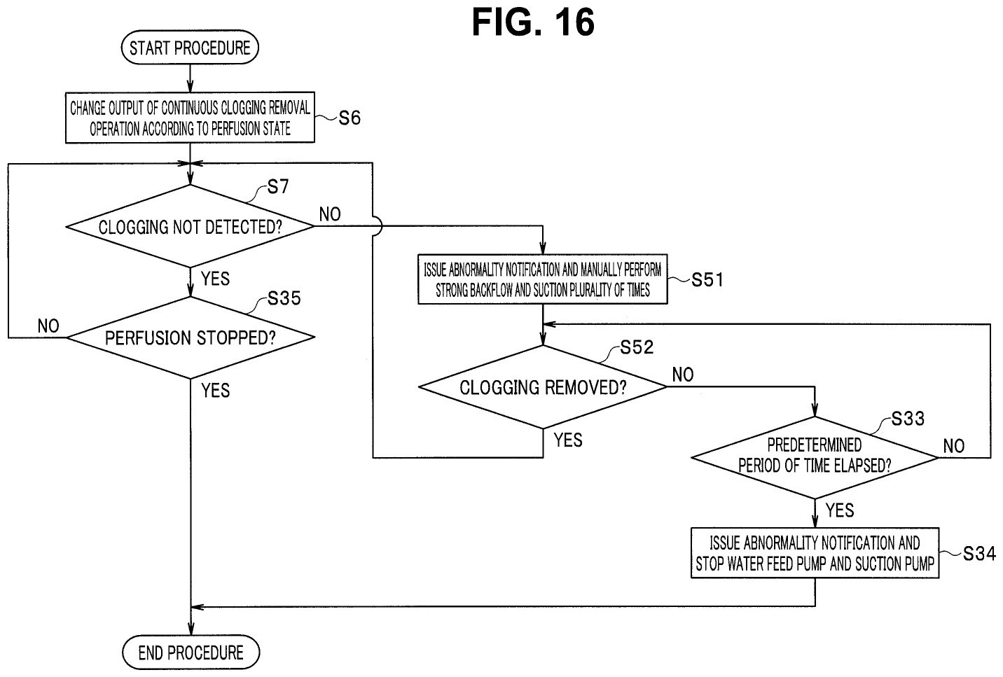

FIG. 16 is a flow chart for explaining another example of a more detailed control method of the perfusion control by the fluid perfusion apparatus 10. In FIG. 16, similar processing to FIG. 14 and FIG. 15 will be denoted by same reference signs and a description of such processing will be omitted.

When the control circuit 11 detects clogging in the processing of step S7, the control circuit 11 automatically generates a strong backflow and suction a plurality of times (S51). For example, the control circuit 11 automatically generates a strong backflow and suction 10 times.

Next, the control circuit 11 determines whether or not the clogging is removed (S52). When the control circuit 11 determines that the clogging is removed (S52: YES), the control circuit 11 returns to the processing of step S7. On the other hand, when the control circuit 11 determines that the clogging is not removed (S52: NO), the control circuit 11 makes a transition to the processing of step S33. Processing of step S33 and thereafter are similar to those shown in FIG. 14.

In the processing shown in FIG. 16, a strong backflow and suction are generated a plurality of times (for example, 10 times) in order to deal with stubborn clogging in advance. In this case, since generating a strong backflow and suction a plurality of times may possibly cause fragmented stones to scatter, desirably, an abnormality notification is concomitantly issued. When the clogging is not removed even after generating a strong backflow and suction a plurality of times, an abnormality is notified in a similar manner to the processing shown in FIG. 14.

While a strong backflow and suction are generated a plurality of times in the processing shown in FIG. 16, control is not limited thereto and may involve repetitively generating a strong backflow and suction over a predetermined period of time or involve continuing to generate a strong backflow and suction until the clogging is removed during the process of repetitively generating the strong backflow and suction over a predetermined period of time.

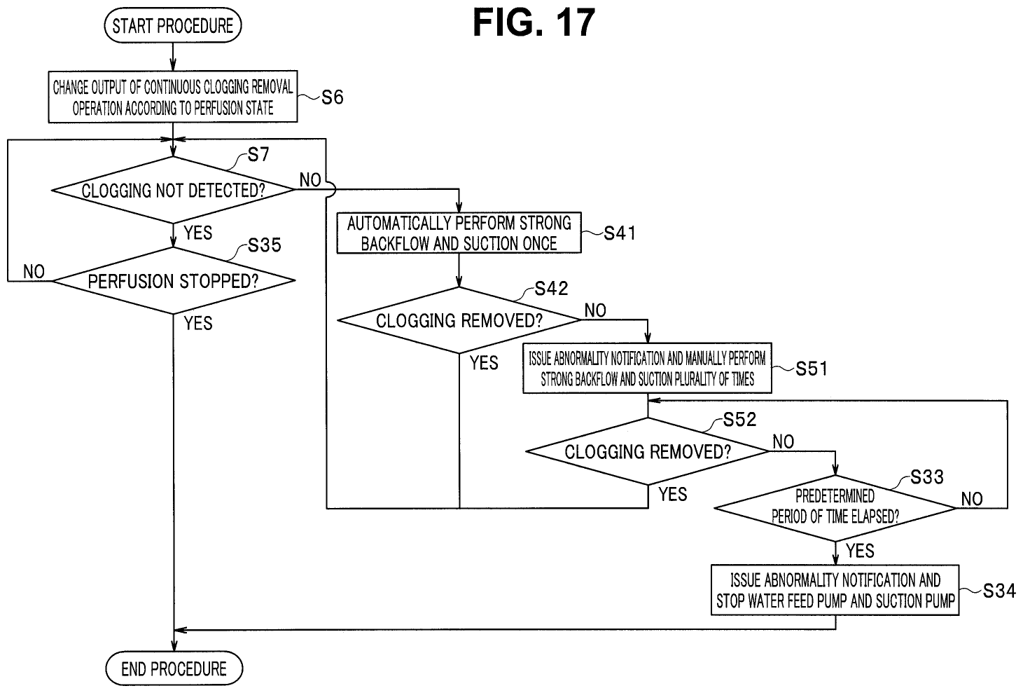

FIG. 17 is a flow chart for explaining another example of a more detailed control method of the perfusion control by the fluid perfusion apparatus 10. In FIG. 17, similar processing to FIG. 14, FIG. 15, and FIG. 16 will be denoted by same reference signs and a description of such processing will be omitted.

When the control circuit 11 detects clogging in the processing of step S7, in step S41, the control circuit 11 automatically generates a strong backflow and suction once.

Next, in step S42, the control circuit 11 determines whether or not the clogging is removed. When the control circuit 11 determines that the clogging is removed (S42: YES), the control circuit 11 returns to the processing of step S7. On the other hand, when the control circuit 11 determines that the clogging is not removed (S42: NO), the control circuit 11 makes a transition to the processing of step S51. Processing of step S51 and thereafter are similar to those shown in FIG. 16.

The processing shown in FIG. 17 is a combination of the processing shown in FIG. 15 with the processing shown in FIG. 16. By such control, a burden on an operator or the like can be reduced and, at the same time, a procedure can be performed in an efficient manner.

### Modifications

While a backflow and suction are generated using the drive mechanism 16 including the syringe 80 and the cam 82 in the first embodiment, a configuration for generating a backflow and suction is not limited thereto.

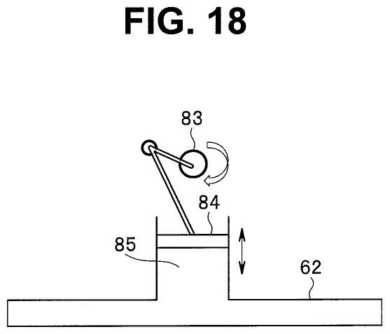

FIG. 18 to FIG. 29 are diagrams showing other examples of the configuration of the drive mechanism 16.

As shown in FIG. 18, the drive mechanism 16 may be a crank mechanism 83. A water storage mechanism 85 is provided midway along a suction conduit and a surface 84 is provided on an upper surface of the water storage mechanism 85. The crank mechanism 83 generates a backflow and suction in the suction conduit by pushing and pulling the surface 84.

In addition, hydraulic power generated by a water wheel may be used instead of electric power to operate such an actuator. By installing the water wheel so as to receive a flow midway along the suction conduit and configuring the actuator such as the crank mechanism 83 for removing clogging to operate under a force of the flow, an operation output or a cycle of the actuator can be changed in accordance with a flow rate.

In addition, in the configuration in which the syringe 80 is pushed and pulled by being fixed to an actuator such as the cam 82 as in the embodiment described above, a fixing mechanism that enables the syringe 80 to be readily detached is desirably included.

The syringe 80 enables both a pushing and pulling operation by an actuator and a manually performed pushing and pulling operation. For example, in addition to removing clogging, the syringe 80 can be used by a helper to arbitrarily generate a backflow in response to a request by an operator for purposes of collecting fragments in a kidney to enable laser fragmentation to be readily performed with a flow of water created by a backflow or blasting away air bubbles or substances adhered to a lens surface of the endoscope 20 to secure a field of view.

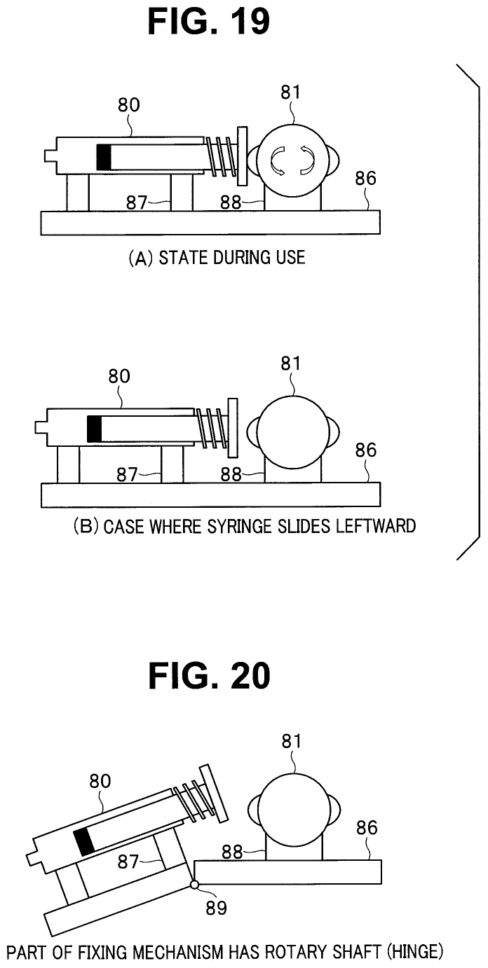

As shown in FIG. 19 (A) and FIG. 19 (B), a fixing mechanism 86 includes a fixing unit 87 configured to fix the syringe 80 and a fixing unit 88 configured to fix the cam 82. While the cam 82 rotates counterclockwise, a rotation direction is not limited to counterclockwise. The fixing unit 87 configured to fix the syringe 80 slides in a longitudinal axis direction of the syringe 80. When detaching the syringe 80 from the fixing mechanism 86, the syringe 80 can be readily detached by sliding the fixing unit 87 and releasing a contact between the syringe 80 and the cam 82. Note that the fixing unit 88 configured to fix the cam 82 may be slid in a longitudinal axis direction of the syringe 80.

In addition, as shown in FIG. 20, the fixing mechanism 86 may include a rotary shaft 89 capable of rotating a part of the fixing mechanism 86. The rotary shaft 89 is constituted of, for example, a hinge.

With such a configuration, since the syringe 80 can be readily attached to and detached from the fixing mechanism 86, a hassle or time when manually using the syringe 80 in accordance with a request by the operator can be reduced.

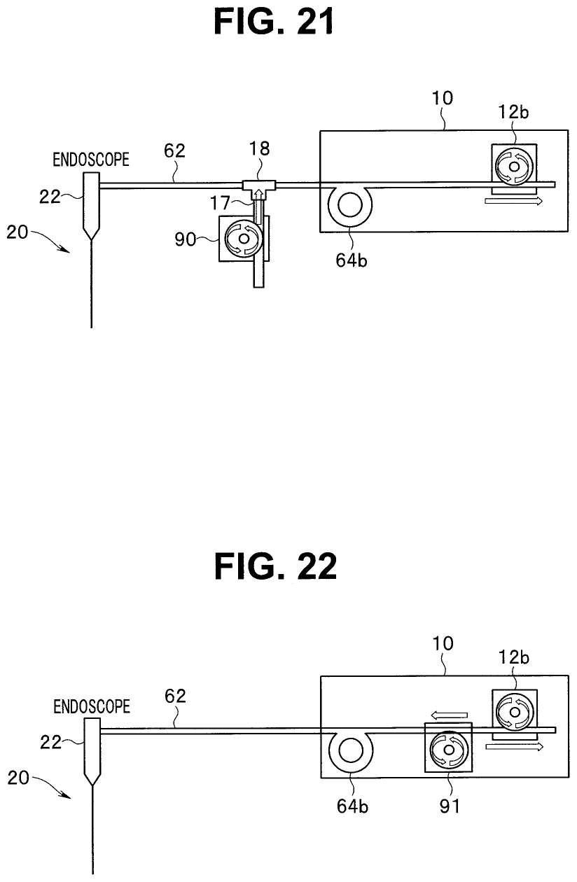

Furthermore, as shown in FIG. 21, a pump 90 instead of the syringe 80 may be connected to the tube 17 being connected by the branch 18 and a backflow and suction may be generated by an operation of the pump 90.

In addition, as shown in FIG. 22, a pump 91 configured to operate in an opposite direction to the suction pump 12b may be arranged between the secondary strainer 64b and the suction pump 12b and a backflow and suction may be generated by an operation of the pump 91. A position where the pump 91 is arranged is not limited to a position shown in FIG. 22 as long as the position is midway along the suction conduit.

The pump 91 configured to operate in an opposite direction is required not to obstruct the suction conduit at timings other than a backflow unless the pump 91 performs suction in a same amount and in a same direction as the suction pump 12b. In addition, in this mode, since operating the pump 91 in the opposite direction while continuing to operate the suction pump 12b causes pressure inside the suction conduit between the suction pump 12b and the pump 91 to drop significantly and causes water to flow into the conduit at the reduced pressure from a side of the endoscope 20 at the moment conduit obstruction by the pump 91 in the opposite direction is released, a strong suction is created inside the suction channel 27.

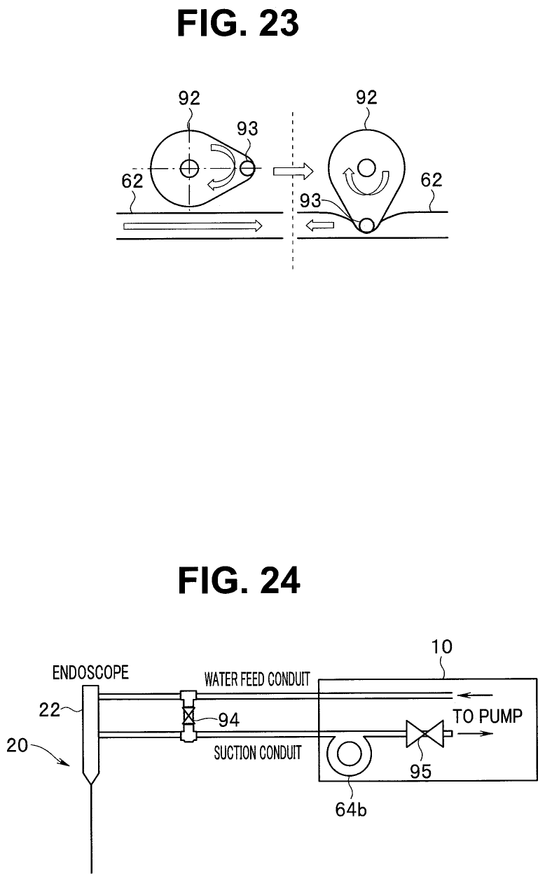

In addition, a backflow may be generated by ironing the suction tube 62 using a cam 92 as shown in FIG. 23 in place of the pump 90 or 91. The cam 92 includes a roller 93 in a projecting portion thereof. With such a configuration, friction when ironing the suction tube 62 can be reduced.

Furthermore, as an embodiment that differs from methods using the syringe 80 or the additional pumps 90 and 91, a backflow and suction may be generated by opening and closing a valve or controlling the suction pump 12b.

For example, a backflow and suction can also be realized by temporarily operating the suction pump 12b in an opposite direction to cause a backflow and, subsequently, immediately operating the suction pump 12b in an original direction at a higher output than an original output.

In addition, as shown in FIG. 24, by temporarily connecting a water feed conduit and a suction conduit in a perfusion to each other with a valve 94, a backflow may be generated by feeding water into the suction conduit from the water feed conduit. In this case, although suction is automatically applied once again by restoring the valve 94, in order to apply stronger suction than the original suction, a strong suction must be applied to the endoscope 20 by a method of temporarily increasing an output of the suction pump 12b or connecting the suction conduit to a conduit which is present in a surgical environment and which is subjected to suction pressure.

To this end, as shown in FIG. 24, a valve 95 configured to block the suction conduit is used separately from the valve 94 configured to cause water to flow into the suction conduit from the water feed conduit. By simultaneously opening the valve 94 and closing the valve 95, water from the water feed conduit only flows toward the side of the endoscope 20 and generates a strong backflow. Subsequently, by simultaneously closing the valve 94 and opening the valve 95, strong suction due to a continuous drop of negative pressure in the suction conduit between the valve 95 and the suction pump 12b is generated.

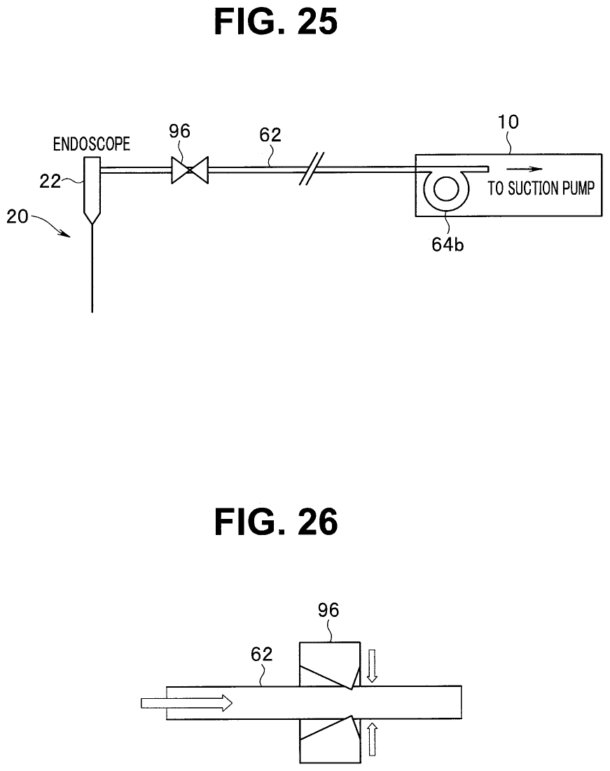

In addition, as shown in FIG. 25, a valve 96 configured to open and close a suction conduit may be provided. Opening and closing the valve 96 also creates a backflow due to a water hammer effect and suction due to a release of dropped pressure.

In doing so, as shown in FIG. 25, a shape of the valve 96 is structured to push water toward the side of the endoscope 20 when being closed. More specifically, an incline of the valve 96 on the side of the endoscope 20 is made more gradual than an incline of the valve 96 on a side of the fluid perfusion apparatus 10. With such a structure, a backflow of water can be generated in addition to the water hammer effect.

In the respective configurations described above, for example, by increasing the output of the suction pump 12b in accordance with a timing of suctioning a fragmented stone inside the suction channel 27 by suction created by the syringe 80, by releasing the valves 95 and 96, or the like, an impact by subsequent fragmented stones in the mechanism of clogging removal can be increased.

In addition, when an apparatus configured to generate positive pressure or negative pressure in an environment of a procedure can be secured, a conduit branched from a suction conduit may be connected to the positive pressure or the negative pressure and a valve may be provided midway along the conduit. Accordingly, by opening the valve of the conduit connected to the positive pressure, a backflow can be generated inside the suction conduit in a similar manner to when pushing the syringe 80. In addition, by opening the valve of the conduit connected to the negative pressure, strong suction can be generated inside the suction conduit in a similar manner to when pulling the syringe 80.

Furthermore, while output of a continuous clogging removal operation is increased by control by the control circuit 11 as a method of generating a strong backflow and suction in processing for clogging removal in the embodiment described above, the method is not limited thereto.

For example, by configuring the drive mechanism 16 so as to include a plurality of cams which push the syringe 80 over different lengths and switching among the plurality of cams, strengths of a backflow and suction may be switched among different settings. In addition, strengths of a backflow and suction may be switched among different settings by changing a positional relationship between the syringe 80 and the cam 82.

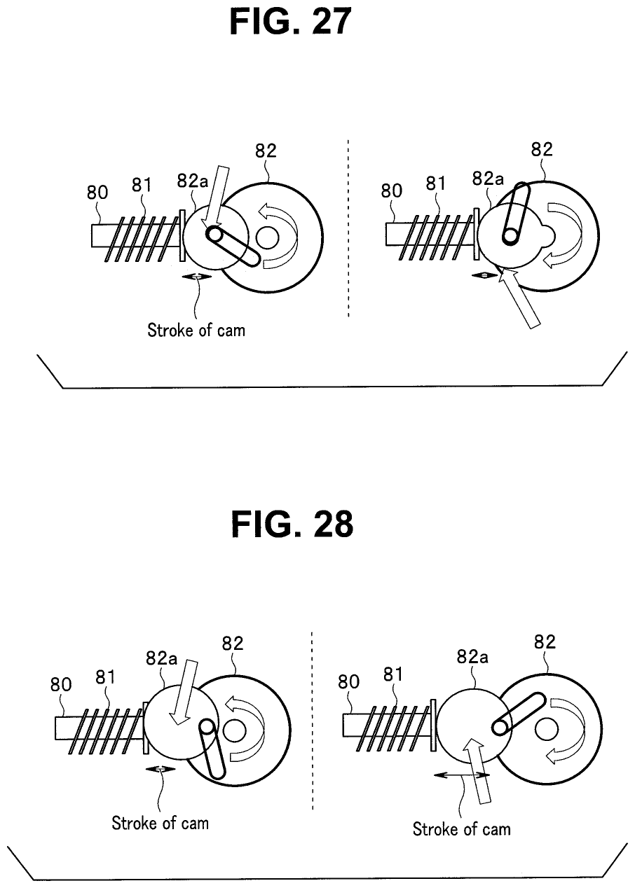

Moreover, as shown in FIG. 27 and FIG. 28, an amount of projection of the projecting portion 82a may be changed in accordance with a rotation direction of the cam 82. For example, by changing the amount of projection of the projecting portion 82a that is constituted of a roller or the like, a pushing amount of the syringe 80 is changed and strengths of a backflow and suction are switched among different settings.

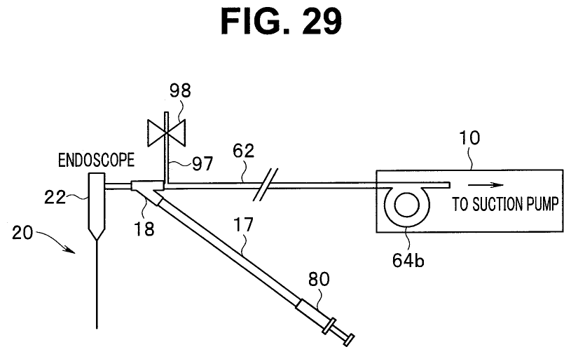

In addition, as shown in FIG. 29, a branch tube 97 connected to atmosphere may be connected to the suction tube 62 and a solenoid valve 98 may be provided in the branch tube 97. The solenoid valve 98 is closed during normal use. When clogging occurs and, at the same time, when a strong backflow is required, the solenoid valve 98 is opened (for example, 0.3 seconds). Accordingly, a water hammer effect can be created inside the suction conduit and a backflow can be generated.

While only the syringe 80 is illustrated in FIG. 29, a drive mechanism such as the cam 82 or a linear actuator may be provided. In addition, a strong backflow may be generated by changing an operation amount of the linear actuator.

### Second Embodiment

Next, a second embodiment will be described.

In the second embodiment, the method of “performing the operation when an occurrence or a development of clogging is detected until the clogging is removed” will be described.

A lower limit of a backflow for “removing clogging when an occurrence or a development of the clogging is detected” must be set to “a sufficient amount of water/flow rate to cause clogged fragments to retreat inside the suction channel 27”.

As the amount of water, when stopping perfusion upon removal of the clogging, a flow of water of 0.2 ml or more may be provided. On the other hand, when removing clogging while continuing the perfusion, since a backflow is generated against the flow of the perfusion, the amount of water becomes larger than the value described above.

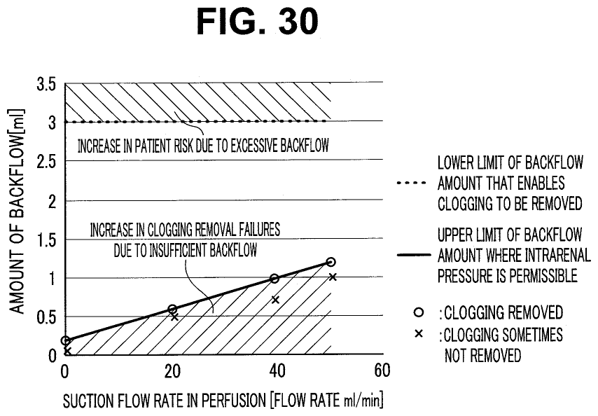

FIG. 30 is a diagram showing a relationship between a flow rate of a backflow that removes clogging and a flow rate of suction by perfusion.

More specifically, as shown in FIG. 30, when an amount of perfusion of the endoscope 20 is set to 10 to 50 ml/min, a lower limit value of a flow of water necessary for removing clogging in both cases is 1.2 ml or more.

On the other hand, if the amount of water of the backflow is excessively large, pressure increases due to a large amount of water flowing into the kidney and may result in an elevated risk of complications or the like of a patient. Based on previous findings, risk can be suppressed if an upper limit value of the flow of water is around 3 ml. As things stand, a flow rate of a backflow is set within a range of 1.2 to 3 ml in consideration of a shortness of time it takes to remove clogging, a magnitude of an amount of force required to push the syringe 80 inward, and a risk to the patient. More preferably, the flow rate of a backflow is optimally set within a range of 1.5 to 2 ml. In addition, it has been found that the higher a frequency of a backflow (the number of times a backflow and suction are repetitively generated per unit time), the more readily clogging may be removed, and a backflow is desirably generated at a frequency of two times per second or higher.

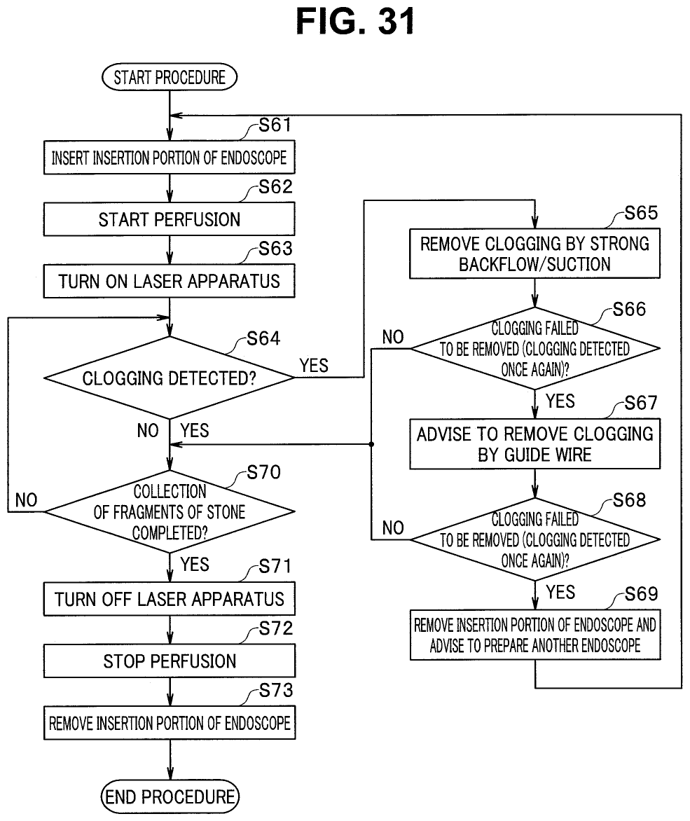

Next, operations of the embodiment configured as described above will be explained with reference to FIG. 31. FIG. 31 is a flow chart for explaining an example of perfusion control by the fluid perfusion apparatus 10 according to the second embodiment.

First, when a procedure is started, the insertion portion 21 of the endoscope 20 is inserted into an organ of a subject (S61). The control circuit 11 controls the water feed pump 12a and the suction pump 12b and starts perfusion (S62).

Next, the laser apparatus 30 is turned on (S63). Then, the distal end of the laser fiber 31 is pointed toward a stone inside the organ and the stone is irradiated with a laser. By irradiating the stone with a laser, the stone is fragmented and becomes fragmented stones to be collected via the suction channel 27b.

Next, the control circuit 11 determines whether or not clogging is detected (S64). The control circuit 11 detects clogging of the suction conduit based on a measurement result of the pressure gauge 15. When the control circuit 11 detects clogging, the control circuit 11 advances to step S65 and performs processing of clogging removal.

In the processing of clogging removal, first, the control circuit 11 performs clogging removal by a strong backflow and suction (S65). The control circuit 11 generates a strong backflow and suction in the suction conduit by, for example, driving the drive mechanism 16.

Next, the control circuit 11 determines whether or not the clogging could not be removed (S66). When the control circuit 11 determines that the clogging could be removed (S66: NO), the control circuit 11 advances to the processing of step S70. On the other hand, when the control circuit 11 determines that the clogging failed to be removed (S66: YES), the control circuit 11 advises to remove the clogging with a guide wire (S67).

Next, the control circuit 11 determines whether or not the clogging could not be removed (S68). When the control circuit 11 determines that the clogging could be removed (S68: NO), the control circuit 11 advances to the processing of step S70. On the other hand, when the control circuit 11 determines that the clogging failed to be removed (S68: YES), the control circuit 11 advises to extract the insertion portion 21 of the endoscope 20 and prepare another endoscope 20 (S69), and returns to step S61.

When the control circuit 11 determines that clogging was not detected in the processing of step S64 (S64: NO) or when the control circuit 11 determines that the clogging could be removed in the processing of step S66 or S68, the control circuit 11 determines whether or not collection of fragments of the stone has been completed (S70).

When the control circuit 11 determines that collection of fragments of the stone has not been completed (S70: NO), the control circuit 11 returns to step S64 and repeats similar processing. On the other hand, when the control circuit 11 determines that collection of fragments of the stone has been completed (S70: YES), the control circuit 11 turns off the laser apparatus 30 (S71).

Next, the control circuit 11 controls the water feed pump 12a and the suction pump 12b and stops perfusion (S72). Finally, the insertion portion 21 of the endoscope 20 is extracted from the organ of the subject (S73) to finish the procedure.

As described above, when the fluid perfusion apparatus 10 detects clogging of a suction conduit based on a measurement result of the pressure gauge 15, the fluid perfusion apparatus 10 can immediately remove the clogging inside the suction conduit by driving the drive mechanism 16 made up of the syringe 80 and the cam 82 and generating a strong backflow and suction.

The present invention is not limited to the embodiments described above and various modifications, alterations, and the like are possible within the scope of the gist of the present invention.

1. A fluid perfusion apparatus, comprising:
a suction conduit for suctioning a fluid from inside a living body;
a suction source connected to the suction conduit and configured to suction the fluid at a first flow velocity via the suction conduit;
a suction control apparatus connected to the suction conduit and configured to control a flow of the fluid suctioned via the suction conduit; and
a control circuit configured to control the suction control apparatus,
wherein the control circuit is configured to perform control for reversing the flow of the fluid that is suctioned via inside the suction conduit by the suction control apparatus, to generate a backflow, after controlling suction at the first flow velocity by the suction source for a predetermined period of time and to cause the suction control apparatus to perform re-suction at a second flow velocity that is greater than the first flow velocity after continuing the backflow for the predetermined period of time.

2. The fluid perfusion apparatus according to claim 1, wherein the suction control apparatus is configured to consecutively perform the backflow and the re-suction at the second flow velocity.

3. The fluid perfusion apparatus according to claim 1, comprising:
a fluid detection apparatus configured to detect a flow rate of the fluid that flows through the suction conduit or an internal pressure of the suction conduit,
wherein the control circuit is configured to control the suction control apparatus and change a magnitude of the backflow and a magnitude of the re-suction at the second flow velocity based on a detection result of the fluid detection apparatus.

4. The fluid perfusion apparatus according to claim 1, wherein
the suction control apparatus is constituted of a syringe connected to the suction conduit, and
the backflow is generated by pushing the syringe and the re-suction at the second flow velocity is generated by pulling the syringe.

5. The fluid perfusion apparatus according to claim 2, wherein
the suction control apparatus is constituted of a syringe connected to the suction conduit and a drive mechanism configured to push the syringe inward, and
the backflow and the re-suction at the second flow velocity are consecutively performed by driving the drive mechanism.

6. A fluid perfusion apparatus, comprising:
a suction conduit for suctioning a fluid from inside a living body;
a suction source connected to the suction conduit and configured to suction the fluid at a first flow velocity via the suction conduit;
a suction control apparatus connected to the suction conduit and configured to control a flow of the fluid suctioned via the suction conduit;
a fluid detection apparatus configured to detect a flow rate of the fluid that flows through the suction conduit or an internal pressure of the suction conduit; and
a control circuit configured to control the suction control apparatus based on information from the fluid detection apparatus,
wherein the control circuit is configured to perform control for reversing the flow of the fluid that is suctioned via inside the suction conduit by the suction control apparatus, to generate a backflow, when it is detected that the flow rate of the fluid is equal to or lower than a specified value or the internal pressure of the suction conduit is equal to or higher than a specified value by the fluid detection apparatus and to cause the suction control apparatus to perform re-suction at a second flow velocity that is greater than the first flow velocity after continuing the backflow for a predetermined period of time.

7. The fluid perfusion apparatus according to claim 6, comprising:
an abnormality notification apparatus configured to notify an abnormality when the fluid detection apparatus detects that the flow rate of the fluid is equal to or lower than a specified value or the internal pressure of the suction conduit is equal to or higher than a specified value.

8. The fluid perfusion apparatus according to claim 7, wherein the abnormality notification apparatus is configured to display the abnormality on a display unit or to notify the abnormality by voice.

9. The fluid perfusion apparatus according to claim 6, wherein
the suction control apparatus is constituted of a syringe connected to the suction conduit, and
the backflow is generated by pushing the syringe and the re-suction at the second flow velocity is generated by pulling the syringe.

10. The fluid perfusion apparatus according to claim 6, wherein
the suction control apparatus is constituted of a syringe connected to the suction conduit and a drive mechanism configured to push the syringe inward, and
the backflow and the re-suction at the second flow velocity are performed by driving the drive mechanism.

11. A fluid perfusion method, comprising:
suctioning a fluid inside a living body at a first flow velocity via a suction conduit;
after suctioning for a predetermined period of time, reversing a flow of a mid fluid that is suctioned via the suction conduit, to generate a backflow;
performing re-suction at a second flow velocity that is higher than the first flow velocity after continuing the backflow; and
performing suction at the first flow velocity after performing the re-suction for a predetermined period of time.

12. The fluid perfusion method according to claim 11, comprising:
consecutively performing the backflow and the re-suction at the second flow velocity.

13. The fluid perfusion method according to claim 11, comprising:
detecting a flow rate of the fluid that flows through the suction conduit or an internal pressure of the suction conduit; and
changing a magnitude of the backflow and a magnitude of the re-suction at the second flow velocity based on the detection result.

14. The fluid perfusion method according to claim 11, comprising:
generating the backflow by pushing a syringe connected to the suction conduit and generating the re-suction at the second flow velocity by pulling the syringe.

15. The fluid perfusion method according to claim 12, comprising:
consecutively performing the backflow and the re-suction at the second flow velocity by driving a drive mechanism and pushing a syringe connected to the suction conduit inward.

16. A fluid perfusion method, comprising:
suctioning a fluid inside a living body at a first flow velocity via a suction conduit;
detecting a flow rate of the fluid that flows through the suction conduit or an internal pressure of the suction conduit; and
reversing a flow of the fluid that is suctioned via inside the suction conduit, to generate a backflow, when it is detected that the flow rate of the fluid is equal to or lower than a specified value or the internal pressure of the suction conduit is equal to or higher than a specified value and performing re-suction at a second flow velocity that is greater than the first flow velocity after continuing the backflow for a predetermined period of time.

17. The fluid perfusion method according to claim 16, comprising:
notifying an abnormality when it is detected that the flow rate of the fluid is equal to or lower than a specified value or the internal pressure of the suction conduit is equal to or higher than a specified value.

18. The fluid perfusion method according to claim 17, comprising:
displaying the abnormality on a display unit or notifying the abnormality by voice.

19. The fluid perfusion method according to claim 16, comprising:
generating the backflow by pushing a syringe connected to the suction conduit and generating the re-suction at the second flow velocity by pulling the syringe.

20. The fluid perfusion method according to claim 16, comprising:
performing the backflow and the re-suction at the second flow velocity by driving a drive mechanism and pushing a syringe connected to the suction conduit inward.
